(19) United States
(12) Patent Application Publication
(10) Pub. No.: US 2014/0303672 A1
(43) Pub. Date: Oct. 9, 2014

(54) SYSTEMS AND METHODS FOR STABILIZING THE SPINE

(71) Applicant: Bao Tran, (US)

(72) Inventors: Bao Tran, Saratoga, CA (US); Ha Tran, Saratoga, CA (US)

(73) Assignee: Bao Tran, Saratoga, CA (US)

(21) Appl. No.: 13/858,615

(22) Filed: Apr. 8, 2013

(51) Int. Cl. A61B 17/70

(52) U.S. Cl. CPC A61B 17/7031
USPC 606/255; 29/592; 29/525.01

(57) ABSTRACT

A device for treatment of spinal deformities or injuries includes: a plurality of rods made of shape memory metal; attachment devices to attach the rods to the vertebrae; and a rod end cover to make it comfortable for the patient.

### BACKGROUND

This invention relates generally to surgical devices for stabilizing the spine, and more particularly to a spinal implant.

The spinal column is a complex system of bones and connective tissue which protects critical elements of the nervous system. Despite these complexities, the spine is a highly flexible structure, capable of a high degree of curvature and twist through a wide range of motion. Trauma or developmental irregularities can result in spinal pathologies which limit this range of motion.

Chronic back problems cause pain and disability for a large segment of the population. In many cases, the chronic back problems are attributed to relative movement between vertebrae in the spine. Spinal surgery includes procedures to stabilize adjacent vertebrae. Common stabilization techniques include fusing the vertebrae together.

For many years, orthopedic surgeons have attempted to correct spinal irregularities and restore stability to traumatized areas of the spine through immobilization. Over the past ten years, spinal implant systems have been developed to achieve immobilization. Spinal surgery devices are placed during surgery and manipulated with clamps or screws. Examples of such systems are disclosed in U.S. Pat. Nos. 5,102,412 and 5,181,917 to Rogozinski. Such systems often include spinal instrumentation having connective structures such as elongated rods which are placed on opposite sides of the portion of the spinal column intended to be immobilized. Screws and hooks are commonly utilized to facilitate segmental attachment of such connective structures to the posterior surfaces of the spinal laminae, through the pedicles, and into the vertebral bodies. These components provide the necessary stability both in tension and compression to achieve immobilization.

Various fastening mechanisms have been provided in the prior art to facilitate securement of screws and hooks to the connective structures of a spinal stabilization system. For example, U.S. Pat. No. 5,257,993 to Asher discloses an apparatus for use in retaining a spinal hook on an elongated spinal rod. The apparatus includes a body extending upwardly from a hook portion and having an open ended recess for receiving a spinal rod and an end cap engageable with the body to close the recess. A set screw is disposed in the center of the end cap to clamp the rod in the recess of the body. The end cap and body are interconnectable by different types of connectors including a bayonet connector, a linear cam connector or a threaded connector. Other examples of fastening mechanism for facilitating attachment of screws and hooks to the connective structures of a spinal stabilization system are disclosed in U.S. Pat. No. 5,437,669 to Yuan et al. and U.S. Pat. No. 5,437,670 to Sherman et al.

U.S. Pat. No. 6,565,565 discloses a device for securing a spinal rod to the spine which includes a head portion configured to receive a spinal rod, a locking cap configured to engage the head portion and the spinal rod upon rotation of the locking cap relative to the head portion to secure the position of the head portion relative to the spinal rod, and a fastener portion depending from the head portion and configured to engage the spine.

There are limitations with stiff materials for spinal surgery. Rods are placed that must be moved into position by clamps or screws. The rods are mostly made of stiff material. Most are off the shelf and not custom to the patient. Many current systems fuse or place fixtures into the spine that do not allow normal movement. The materials are stiff or they limit movement.

### SUMMARY

In one aspect, a device for treatment of spinal deformities or injuries includes: a plurality of rods made of shape memory metal; attachment devices to attach the rods to the vertebrae; and a rod end cover to make it comfortable for the patient.

In another aspect, a method to form device for use with vertebrae includes scanning a patient to obtain 3D patient data; generating a custom spinal device based on the 3D patient data using computer aided design (CAD) tool; and fabricating the custom device using computer controlled equipment.

Implementations of the method can include one or more of the following. The system can fabricate a plurality of rods made of shape memory metal. The attachment devices can be made of shape memory metal to attach rods to the vertebrae. A plurality rod end covers can be done. The rods can be made of shape memory metal; one or more attachment devices to attach the rods to the vertebrae; and a rod end cover. The rods are placed on either side of the vertebrae by the attachment devices and wherein the caps are placed on the end of last rods. The rods are placed along the vertebrae with attachment devices and allowed to slide during movement. Two or more segments of rods can be fabricated corresponding to bones that need to be aligned. Each segment comprises different material properties based on temperature when device is placed. An operator can surgically install the device in a patient by altering the shape of the rod prior to installation and allowing the patient heat to change the shape of the rod after installation.

Advantages of the system may include one or more of the following. Using 3D imaging and software techniques, shape memory alloy, can be used and be custom made for individual patients. A more flexible rod made from a shape memory alloy and custom made offers the advantage of more range of movement and lower forces to the patient.

### BRIEF DESCRIPTION OF THE DRAWINGS

FIG. 1 shows an exemplary process for forming a customized spinal treatment device.

FIG. 2 shows an exemplary process for deploying the device formed in FIG. 1.

FIG. 3 shows an exemplary spinal treatment device.

### DESCRIPTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features. However, any single inventive feature may not address any of the problems discussed above or may only address one of the problems discussed above. Further, one or more of the problems discussed above may not be fully addressed by any of the features described below.

Broadly, an embodiment of the present invention utilizes computer software, shape memory alloy that when manufactured can be any shape (curved or straight). When cooled to a certain temperature the device can be manipulated and bent, to conform to a patient's spinal problem. When the device warms to body temperature it returns to its manufactured state. The system offers the advantage of lighter forces and greater flexibility in spinal surgeries.

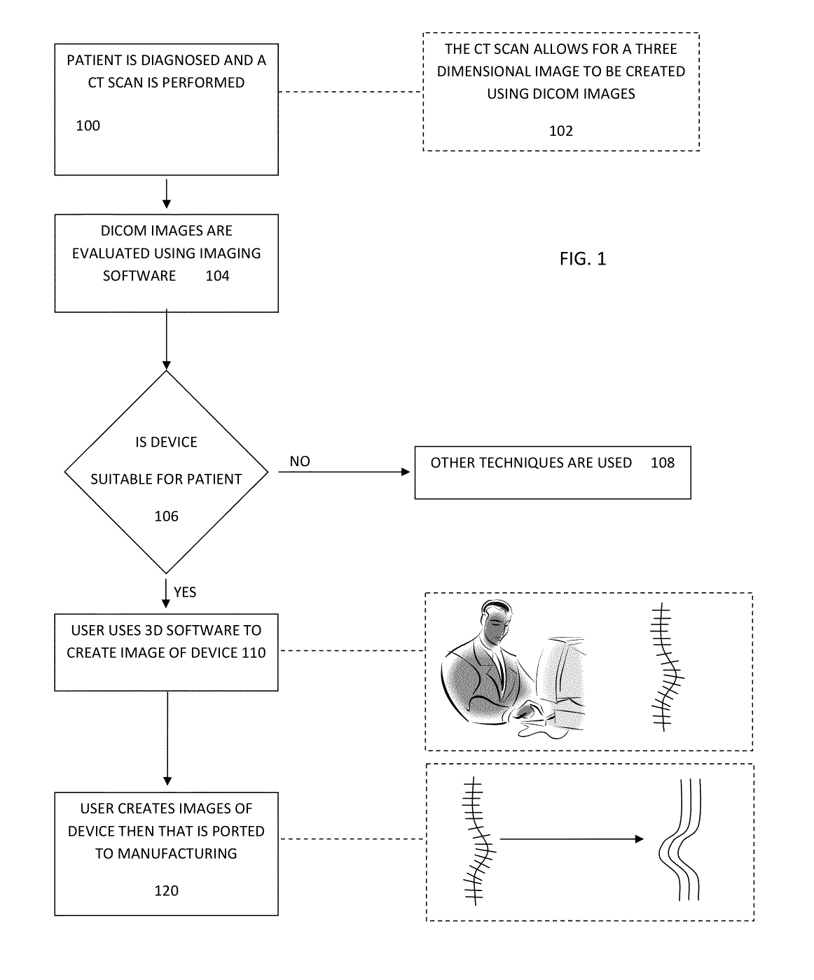

Referring now to FIG. 1, a patient is diagnosed and a CT scan is performed (100). Computed tomography (CT) is a medical imaging method employing tomography created by computer processing. Digital geometry processing is used to generate a three-dimensional image of the inside of an object from a large series of two-dimensional X-ray images taken around a single axis of rotation. CT produces a volume of data which can be manipulated, through a process known as “windowing”, in order to demonstrate various bodily structures based on their ability to block the X-ray/Rontgen beam. Although historically the images generated were in the axial or transverse plane, orthogonal to the long axis of the body, modern scanners allow this volume of data to be reformatted in various planes or even as volumetric (3D) representations of structures. Instead of finding the outline of bones and organs, a CAT scan machine forms a full three-dimensional computer model of a patient's insides. Doctors can even examine the body one narrow slice at a time to pinpoint specific areas such as the spinal regions of the patient.

The CT scan allows for three dimensional images to be created such as Digital Imaging and Communications in Medicine (DICOM) images (102). DICOM is a standard for handling, storing, printing, and transmitting information in medical imaging. It includes a file format definition and a network communications protocol. The communication protocol is an application protocol that uses TCP/IP to communicate between systems. DICOM files can be exchanged between two entities that are capable of receiving image and patient data in DICOM format. The National Electrical Manufacturers Association (NEMA) holds the copyright to this standard. It was developed by the DICOM Standards Committee, whose members are also partly members of NEMA. DICOM enables the integration of scanners, servers, workstations, printers, and network hardware from multiple manufacturers into a picture archiving and communication system (PACS). The different devices come with DICOM conformance statements which clearly state the DICOM classes they support. DICOM has been widely adopted by hospitals and is making inroads in smaller applications like dentists' and doctors' offices. DICOM is known as NEMA Standard PS3, and as ISO Standard 12052.

Part 10 of the standard describes a file format for the distribution of images. This format is an extension of the older NEMA standard. Most people refer to image files which are compliant with Part 10 of the DICOM standard as DICOM format files. A single DICOM file contains both a header (which stores information about the patient's name, the type of scan, image dimensions, etc), as well as all of the image data (which can contain information in three dimensions). This is different from the popular Analyze format, which stores the image data in one file (*.img) and the header data in another file (*.hdr). Another difference between DICOM and Analyze is that the DICOM image data can be compressed (encapsulated) to reduce the image size. Files can be compressed using lossy or lossless variants of the JPEG format, as well as a lossless Run-Length Encoding format (which is identical to the packed-bits compression found in some TIFF format images).

DICOM is the most common standard for receiving scans from a hospital. Neuroimagers and neuropsychologists who wish to use SPM to normalize scans to stereotaxic space will need to convert these files to Analyze format. Software such as MRIcro, Medcon and XMedcon software will directly convert most DICOM images to and from Analyze format.

The DICOM images are evaluated using suitable imaging software (104). A professional user such as a doctor makes a decision on whether devices such as shape memory rods can be used (106). If not, conventional treatment options can be used (108).

Alternatively, if the shape memory rods can be used, the user creates a specification of the device (110), including 3D shape or image of the device that best fits the patient. A physician would diagnose a spinal condition, for example, scoliosis. A CT scan would be taken as part of the diagnostic procedure, and the physician can provide a prescription that helps the patient, and an operator can convert the prescription into a mechanical specification. For example, the images can be converted into 3D structures in computer space, and a user can design a rod that is custom fitted to the patient's body structure using CAD/CAM programs such as Autocad or other suitable CAD tools.

The 3D shapes are then transferred to a manufacturing machine such as a mass customization machine for manufacturing (112). In one embodiment, a milling machine can be used for the shaping of metal and other solid materials. Milling machines exist in two basic forms: horizontal and vertical, which terms refer to the orientation of the cutting tool spindle. Unlike a drill press, in which the workpiece is held stationary and the drill is moved vertically to penetrate the material, milling also involves movement of the workpiece against the rotating cutter, the latter of which is able to cut on its flanks as well as its tip. Workpiece and cutter movement are precisely controlled to less than 0.001 inches (0.025 millimeters), usually by means of precision ground slides and leadscrews or analogous technology. Milling machines can perform a vast number of operations, some very complex, such as slot and keyway cutting, planning, drilling, diesinking, rebating, routing, etc. Cutting fluid is often pumped to the cutting site to cool and lubricate the cut, and to sluice away the resulting swarf.

Milling machines may be manually operated, mechanically automated, or digitally automated via computer numerical control (CNC). In the CNC system, end-to-end component design is highly automated using CAD/CAM programs. The programs produce a computer file that is interpreted to extract the commands needed to operate a particular machine, and then loaded into the CNC machines for production. Since any particular component might require the use of a number of different tools—drills, saws, etc.—modern machines often combine multiple tools into a single “cell”. In other cases, a number of different machines are used with an external controller and human or robotic operators that move the component from machine to machine. In either case, the complex series of steps needed to produce any part is highly automated and produces a part that closely matches the original CAD design.

The CNC software handles motion control which includes sampling the position of the axes to be controlled, computing the next point on the trajectory, interpolating between these trajectory points, and computing an output to the motors. For servo systems, the output is based on a PID compensation algorithm. For stepper systems, the calculations run open-loop, and pulses are sent to the steppers based on whether their accumulated position is more than a pulse away from where their commanded position should be. The motion controller includes programmable software limits, interfaces to hardware limit and home switches, PID servo compensation with zero, first, and second order feedforward, maximum following error, selectable velocity and acceleration values, individual axis jogging (continuous, incremental, absolute), queued blended moves for linear and generalized circular motion, and programmable forward and inverse kinematics. Initialization files (with the same syntax as Microsoft Windows INI files) are used to configure parameters such as number and type of axes (e.g., linear or rotary), scale factors between feedback devices (e.g., encoder counts) and axis units (e.g., millimeters), servo gains, servo and trajectory planning cycle times, and other system parameters. Complex kinematics for robots can be coded in C according to a prescribed function interface and linked in to replace the default 3-axis Cartesian machine kinematics routines. When controlling actual machines, the motion controller requires a real-time operating system. The motion controller uses either shared memory or RT-Linux FIFOs to receive commands or send status, error, or logging information.

Although CNC can be used, other alternatives are contemplated as well. For example, a stereo lithography approach can be used. Stereolithography is an additive manufacturing process using a vat of liquid UV-curable photopolymer “resin” and a UV laser to build parts a layer at a time. On each layer, the laser beam traces a part cross-section pattern on the surface of the liquid resin. Exposure to the UV laser light cures, or, solidifies the pattern traced on the resin and adheres it to the layer below. After a pattern has been traced, the SL's elevator platform descends by a single layer thickness, typically 0.05 mm to 0.15 mm (0.002″ to 0.006″). Then, a resin-filled blade sweeps across the part cross section, re-coating it with fresh material. On this new liquid surface, the subsequent layer pattern is traced, adhering to the previous layer. A complete 3-D part is formed by this process. After building, parts are cleaned of excess resin by immersion in a chemical bath and then cured in a UV oven.

In one embodiment, the rod can be formed directly through stereolithography. This rod will be of a plastic material and will conform to a predetermined shape even if bent during operation. In another embodiment, the 3D part can be the negative of the desired rod shape. The rod metal can be poured into the negative mold and allowed to cool to form the custom shape.

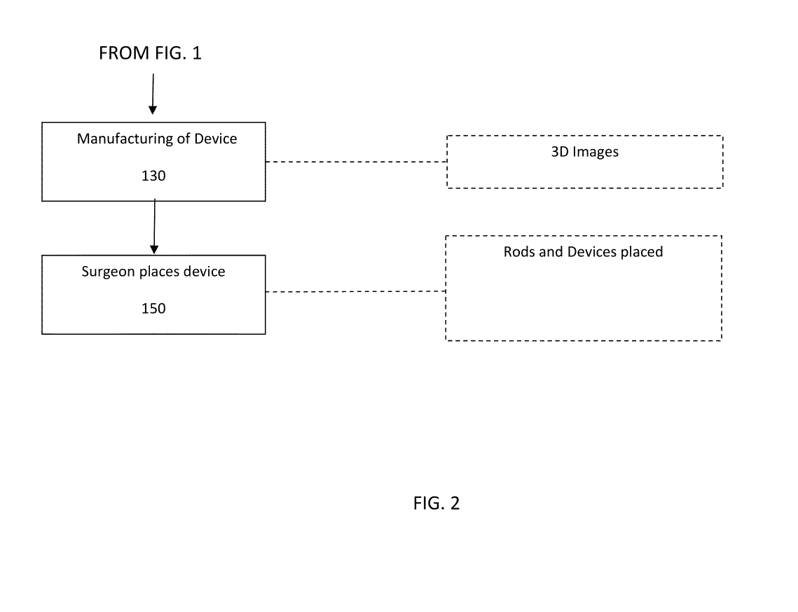

FIG. 2 shows an exemplary installation process. The device is designed as in FIG. 1, and the device is manufactured using a mass customization equipment such as CNC machine, among others (130). Next, a surgeon can place the device in the patient (150). In an embodiment, to use the present invention, a CT scan of the patient is obtained. A 3D rendering is performed with a 3 dimensional model of the spinal column. The CT scan is used for diagnosis and for manufacturing the rod to be placed or in manufacturing specific conformations of the rod to be placed in the patient. The rods would be surgically placed in a patient's back anchored by surgical screws or other attachment devices with caps placed at the end of the rods.

Shape memory material is formed to the spinal column utilizing the physicians input regarding where the device should go. The rod is bent or left straight for the patient, depending on the condition. Then it is sterilized and packaged.

In an embodiment, rods and attachment devices can be custom made depending on outcome of computer scan, or off the shelf attachment devices can be used with the custom rods. Shape memory material could be used in building to make them more vibration resistant, making the structure more flexible.

The computer fabricated device for securing a spinal rod to the spine includes a head portion having a channel extending therethrough configured to receive a spinal rod, a locking cap including a first portion configured to engage an interior surface of the head portion and a second portion configured to engage an exterior surface of a spinal rod received by the channel to secure the position of the head portion relative to the spinal rod, and a fastener portion depending from the head portion and configured to engage the spine.

An embodiment of the device includes two or more computer fabricated rods that fit the patient's body (Element A), surgical screws and attachment devices (Element B) to attach rods to the vertebrae and a rod end unit (Element C) to allow the ends of rods to be capped and comfortable to patient.

In an embodiment, element A is preferably the length of the spine (but not necessarily) between about 1 and 3 feet in length. It includes a rod made from shape memory metal and preferably (but not necessarily) 0.2-0.5 inches in diameter. It can be other shapes in cross section. Element B consists of surgical screws and fixtures of various shapes and dimensions to hold rod to vertebrae. Element C includes a cap which can allow the rod to slide in and out of and conveys a level of comfort.

The shape memory alloy rod allows for easier physician manipulation during placement and better patient adaptation. The attachment devices that hold the rod against the vertebra can vary in size and shape. The rod cap may be an option. Its purpose is to cover the end of the rod. Making it more comfortable and possibly helping with movement of the rod.

The rod is connected by the surgical screws and attachments to all vertebrae that are affected by condition but not limited to those vertebrae affected. The rods are placed on either side of the vertebrae by the attachment devices and caps are placed on the ends.

The rods are placed along the vertebrae with attachment devices and allowed to slide during movement. The rods which are made from a shape memory allow for great patient flexibility and range of motion. Each cap 230 covers one end of the rod.

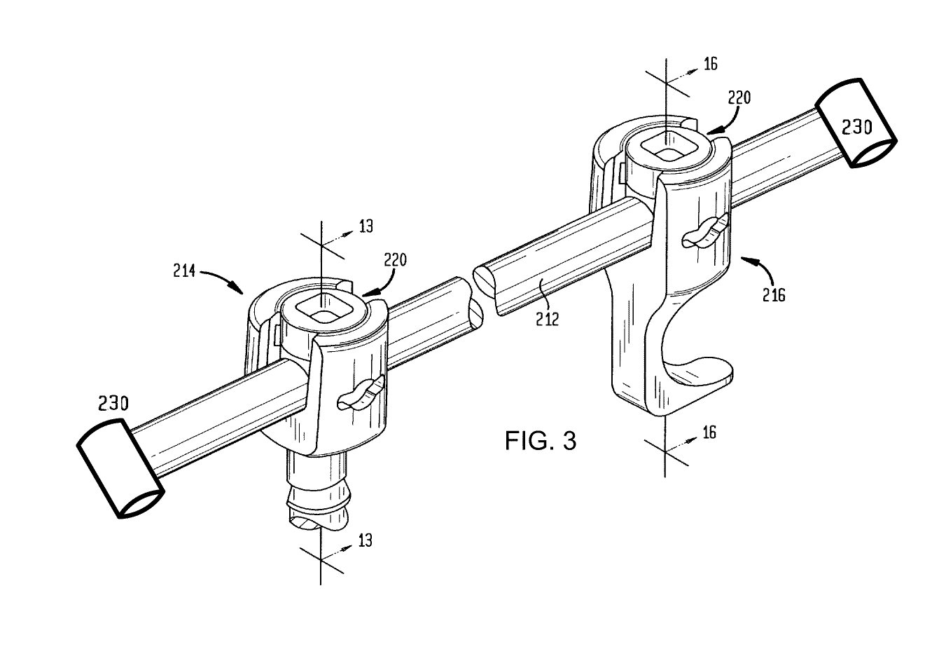

Referring now to FIG. 3, one embodiment of the computer fabricated rods is shown. In FIG. 3, element A includes rods 212, while element B includes screws 220 or screw/hook combination 214/216 can be used. Element C includes caps 230. In the embodiment of FIG. 3, a multi-axial bone screw 214 and a right-angle hook 216 are provided for securing spinal rod 212 to the spine during a spinal stabilization procedure. Both fastening devices employ a top loaded two-piece locking cap, designated generally by reference numeral 220. The two-piece locking cap increases reliability and the ease in which it is installed during a spinal stabilization procedure. While the two-piece locking cap illustrated in FIG. 3 is employed with a multi-axial bone screw, it is readily apparent that the same two-piece locking cap could be employed with a fixed axis bone screw.

The device can include a standard head portion configured to receive a customized spinal rod fabricated in accordance with the patient's body specifics, a locking cap configured to engage the head portion and the spinal rod upon rotation of the locking cap relative to the head portion to secure the position of the head portion relative to the spinal rod, and a fastener portion extending from the head portion and configured to engage the spine. The fastener portion of the device can be in the form of a screw, hook or clamp, or any other configuration known in the art.

In one embodiment, the head portion of the device has a channel extending therethrough for receiving a spinal rod and the channel is preferably bounded by opposed side walls each having an arcuate engagement slot defined therein. The locking cap can have opposed arcuate engagement flanges configured for reception in the opposed arcuate engagement slots of the head portion upon rotation of the locking cap relative to the head portion. Preferably, the opposed engagement slots are each defined in part by inclined slot surfaces, with the angle of the inclined surface of one engagement slot being opposite that of the opposed engagement slot. Similarly, the opposed engagement flanges are preferably each defined in part by inclined flange surfaces, with the angle of the inclined surface of one engagement flange being opposite that of the opposed engagement flange. The head portion also preferably includes structure for interacting with the locking cap to prevent the opposed side walls of the head portion from expanding radially outwardly when the arcuate flanges are engaged in the arcuate slots.

Preferably, the locking cap of the device is configured for rotation between an initial position in which the arcuate engagement flanges are 90° out of phase with the arcuate engagement slots, an intermediate position in which the arcuate engagement flanges are 45° out of phase with the arcuate engagement slots, and a locked position in which the arcuate engagement flanges are in phase and intimately engaged with the arcuate engagement slots.

In this regard, the bottom surface of the locking cap preferably includes a first recess oriented to accommodate a spinal rod when the locking cap is in an initial unlocked position, a second recesses which intersects the first recess at a first angle to accommodate a spinal rod when the locking cap is in an intermediate position, and a third recess which intersects the elongate recess at a second angle to accommodate a spinal rod when the locking cap is in a final locked position. In accordance with a preferred embodiment of the subject disclosure, the first recess is an elongate recess, the second recess is a transverse recess which intersects the elongate recess at a 45° angle, and the third recess is an orthogonal recess which intersects the elongate recess at a 90° angle.

In another embodiment, the locking cap is a two-piece structure which includes an upper portion configured to engage an interior surface of the head portion and a lower portion configured to engage an exterior surface of the spinal rod to secure the position of the head portion relative to the spinal rod upon rotation of the upper portion relative to the lower portion and the head portion. The upper portion of the locking cap includes a bottom surface having an axial reception bore defined therein and the lower portion of the locking cap includes an upper surface having an axial post extending therefrom configured to engage the axial reception bore in the bottom surface of the upper portion of the locking cap and facilitate the relative rotation of the two parts. The upper portion further includes opposed arcuate engagement flanges configured for cammed engagement in correspondingly configured opposed arcuate engagement slots formed in the opposed side walls of the head portion upon rotation of the upper portion relative to the lower portion. The lower portion further includes a bottom surface having an elongated hemi-cylindrical recess that is oriented to accommodate a spinal rod extending through the channel in the head portion.

In accordance with another aspect, the fastener portion is formed monolithic with the head portion, as controlled by a computer so that the fastener/head portion conforms to the patient's body for better fit.

In yet another embodiment, the fastener portion is mounted for movement relative to the head portion. In this regard, the head portion defines a central axis oriented perpendicular to the spinal rod channel and the fastener portion is mounted for angular movement relative to the central axis of the head portion. More particularly, the fastener portion includes a generally spherical head and a threaded body which depends from the spherical head, and the head portion defines a seat to accommodate the spherical head and an aperture to accommodate the threaded body. In use, upon rotation of the upper portion of the locking cap relative to the lower portion of the locking cap into a locked position, the position of the head portion relative to the spinal rod and the position of the fastener relative to the head portion become fixed.

It should be recognized that the subject disclosure is not limited in any way to the illustrated bone screw and right-angle hook. Rather, these particular fasteners are merely examples of the type of devices that can employ the novel locking cap disclosed herein. Other fasteners commonly utilized in spinal stabilization systems, such as, for example, hooks having alternative angular geometries as well as clamps are also envisioned. Indeed, it is envisioned that any component designed for attachment to an elongated spinal rod or transverse coupling rod, may incorporate the novel locking cap of the subject disclosure. Also, any number of fastening devices can be applied along the length of the spinal rod.

An embodiment of the invention can be used where two or more segments of bone need to be aligned and require flexibility and changes of material properties based on temperature when device is placed.

An embodiment of the invention can help with broken bones, trauma, or other types of surgery but also building design or transportation design.

It should be understood, of course, that the foregoing relates to exemplary embodiments of the invention and that modifications may be made without departing from the spirit and scope of the invention as set forth in the following claims.

1. A method to form device for use with vertebrae, comprising:
scanning a patient anatomy using a three dimensional (3D) scanner to obtain 3D patient data;
generating a custom spinal device based on the 3D patient data using computer aided design (CAD) tool; and
facilitating the custom device using computer controlled equipment.

2. The method of claim 1, comprising fabricating a plurality of rods made of shape memory metal.

3. The method of claim 1, comprising fabricating one or more attachment devices made of shape memory metal to attach rods to the vertebrae.

4. The method of claim 1, comprising fabricating a plurality rod end covers.

5. The method of claim 1, comprising fabricating a plurality of rods made of shape memory metal; one or more attachment devices to attach the rods to the vertebrae; and a rod end cover.

6. The method of claim 5, wherein the rods are placed on either side of the vertebrae by the attachment devices and wherein the caps are placed on the end of last rods.

7. The method of claim 5, wherein the rods are placed along the vertebrae with attachment devices and allowed to slide during movement.

8. The method of claim 1, comprising fabricating two or more segments of rods corresponding to bones that need to be aligned.

9. The method of claim 8, wherein each segment comprises different material properties based on temperature when device is placed.

10. The method of claim 1, comprising surgically installing the device in a patient by altering the shape of the rod prior to installation and allowing the patient heat to change the shape of the rod after installation.

11. A system for use with vertebrae, comprising:
a three dimensional (3D) scanner to scan a patient and generate 3D anatomical data of the patient body;
a computer controlled machine to fabricate a spinal device having:
a plurality of rods made of shape memory metal, wherein the rods are custom fabricated to be custom-fitted to the 3D anatomical data;
attachment devices to attach the rods to the vertebrae; and
a rod end cover.

12. The system of claim 11, wherein the 3D scanner comprises a CT-scanner.

13. The system of claim 11, comprising one or more attachment devices made of shape memory metal to attach rods to the vertebrae.

14. The system of claim 11, comprising a plurality rod end covers.

15. The system of claim 11, comprising imaging software to evaluate patient scan data and CAD software to design the custom rods.

16. The system of claim 15, wherein the rods are placed on either side of the vertebrae by the attachment devices and wherein the caps are placed on the end of last rods.

17. The system of claim 15, wherein the rods are placed along the vertebrae with attachment devices and allowed to slide during movement.

18. The device of claim 11, wherein two or more segments of rods support bones that need to be aligned.

19. The system of claim 18, wherein each segment comprises different material properties based on temperature when device is placed.

20. The system of claim 11, wherein the device is surgically positioned in a patient by altering the shape of the rod prior to installation and allowing the patient heat to change the shape of the rod after installation.
